(19) United States
(12) Patent Application Publication
(10) Pub. No.: US 2024/0254422 A1
(43) Pub. Date: Aug. 1, 2024

(54) DEVICES, SYSTEMS, AND METHODS FOR AUTOMATED ASEPTIC SAMPLING

(71) Applicant: IDEX Health & Science, LLC, Rohnert Park, CA (US)

(72) Inventors: Quan Liu, Petaluma, CA (US); Eric Beemer, Anacortes, WA (US); Craig Love, Santa Rosa, CA (US)

(73) Assignee: IDEX Health & Science, LLC, Rohnert Park, CA (US)

(21) Appl. No.: 18/400,078

(22) Filed: Dec. 29, 2023

Related U.S. Application Data
(60) Provisional application No. 63/441,336, filed on Jan. 26, 2023.
(60) Provisional application No. 63/452,543, filed on Mar. 16, 2023.

(51) Int. Cl. C12M 1/26; C12M 1/00; C12M 1/34

(52) U.S. Cl. CPC C12M 33/04; C12M 23/40; C12M 29/20; C12M 33/14; C12M 41/36; C12M 41/40

(57) ABSTRACT

A method for sampling a liquidous material with a sampling system includes providing the sampling system having a gas/liquid separator. The gas/liquid separator includes a separation chamber having a membrane separating the separation chamber into a retentate portion and a permeate portion. The membrane is gas-permeable and liquid impermeable. The gas/liquid separator includes a pressure regulation system fluidically connected to the permeate portion of the separation chamber. The pressure regulation system is configured to control a gas pressure in the permeate portion. The method includes selectively operating the pressure regulation system in a first mode and a second mode. The first mode establishes a first gas pressure condition in the permeate portion of the separation chamber configured to draw a sample of the liquidous material from a reservoir through a first flow channel to the retentate portion of the separation chamber. The second mode establishes a second gas pressure condition in the permeate portion of the gas/liquid separation chamber to push the sample from the retentate portion of the separation chamber through the first flow channel toward the reservoir.

### BACKGROUND

Cell culture is a common technique in the life sciences. To monitor growth or characteristics of biological matter, samples are drawn from a culture (also referred to as a “bioreactor” and/or “reservoir”) and sent to an analyzer to test or measure characteristics of the sample. Various systems are known for delivering biological samples from a bioreactor to an analyzer. In general, these systems include a flow path, i.e., tubes configured to transport fluids and/or gas, and a pump configured to urge the sample from the bioreactor to the analyzer. In order to reduce cross-contamination or carry-over from different samples, there exists a need to pull samples aseptically and clean the flow path. In some cases, analysis of the sample may require a precise amount of sample to be dispensed from the bioreactor.

### SUMMARY

According to some embodiments of the present disclosure, a method for sampling a liquidous material with a sampling system includes providing the sampling system having a gas/liquid separator. The gas/liquid separator includes a separation chamber having a membrane separating the separation chamber into a retentate portion and a permeate portion. The membrane may be gas-permeable and liquid impermeable. The gas/liquid separator includes a pressure regulation system fluidically connected to the permeate portion of the separation chamber. The pressure regulation system is configured to control a gas pressure in the permeate portion. The method includes selectively operating the pressure regulation system in a first mode and a second mode. The first mode establishes a first gas pressure condition in the permeate portion of the separation chamber configured to draw a sample of the liquidous material from a reservoir through a first flow channel to the retentate portion of the separation chamber. The second mode establishes a second gas pressure condition in the permeate portion of the separation chamber to push the sample from the retentate portion of the separation chamber through the first flow channel toward the reservoir.

According to some embodiments of the present disclosure, a method of sampling a liquidous material includes providing a sampling system having a gas/liquid separator. The gas/liquid separator includes a separation chamber having a retentate portion and a permeate port separated by a membrane. The membrane is gas-permeable and impermeable to the liquidous material. The gas/liquid separator includes a pressure regulation system configured to control a pressure in the permeate portion. The method includes providing a first flow channel. The first flow channel fluidically connects the separation chamber to a reservoir containing the liquidous material. The method includes operating the pressure regulation system to impart a negative gas pressure in the permeate portion. The negative gas pressure is effective to pull a sample of the liquidous material from the reservoir to the retentate portion of the separation chamber. The negative gas pressure in the permeate portion is effective to remove a gas from the sample through the membrane.

According to some embodiments of the present disclosure, a sampling system includes a reservoir containing a liquidous material. The sampling system includes a gas/liquid separator including a separation chamber and a pressure regulation system. The separation chamber includes a retentate portion, a permeate portion, and a membrane separating the retentate portion and the permeate portion. The membrane is gas-permeable and liquid impermeable. The pressure regulation system is configured to control a gas pressure in the permeate portion. The sampling system includes a first flow channel fluidically connecting the reservoir to the retentate portion of the gas/liquid separator. The sampling system includes a multi-port selection valve and a displacement pump. A second flow channel fluidically connects the displacement pump to the multi-port selection valve. The multi-port selection valve is selectively adjustable among a plurality of positions, with a first position fluidically connecting the first flow channel to the second flow channel. The pressure regulation system is configured to selectively effect a negative pressure in the permeate portion. The negative pressure in the permeate portion is effective to pull a sample of the liquidous material from the reservoir to the retentate portion of separation chamber. The pressure regulation system is configured to selectively effect a positive pressure in the permeate portion. The positive pressure in the permeate portion is effective to push the sample of liquidous material from the retentate portion of the separation chamber to the reservoir.

According to some embodiments, a sampling system includes a sample probe configured to receive a liquidous material. The sampling system includes a gas/liquid separator including a separation chamber, and a vacuum source fluidically connected to the separation chamber. The sampling system includes a flow path capable of fluidically connecting the sample probe to the gas-liquid separator. A pressurized air source is connectable to the flow path. The gas/liquid separator is operable in a first condition to generate a negative pressure condition in the flow path to urge the liquidous material in a first direction along the flow path.

According to some embodiments, a method of sampling a liquidous material from a reservoir with a sampling system includes providing a sampling system. The sampling system includes a flow path, a gas/liquid separator, multiple port selection valve including a plurality of ports, and a pressurized air source. The gas/liquid separator is fluidically connected to the flow path to generate a negative pressure condition in the flow path. The negative pressure condition urges the liquidous material to move in a first direction along the flow path. The pressurized air source is fluidically connected to the flow path to generate a positive pressure condition in the flow path. The positive pressure condition urges the liquidous material to move in the first direction along the flow path.

### BRIEF DESCRIPTION OF DRAWINGS

This written disclosure describes illustrative embodiments that are non-limiting and non-exhaustive. Reference is made to illustrative embodiments that are depicted in the figures, in which:

FIG. 1A illustrates a diagrammatic view of an exemplary aseptic sampling device, according to some embodiments.

FIG. 1B illustrates a schematic diagram of a control system communicatively connected to an aseptic sampling system, according to some embodiments.

FIG. 2A illustrates a diagrammatic view of an exemplary aseptic sampling device in a first step of sampling, according to some embodiments.

FIG. 2B illustrates a diagrammatic view of an exemplary aseptic sampling device in a second step of sampling, according to some embodiments.

FIG. 2C illustrates a diagrammatic view of an exemplary aseptic sampling device in a third step of sampling, according to some embodiments.

FIG. 2D illustrates a diagrammatic view of an exemplary aseptic sampling device in a fourth step of sampling, according to some embodiments.

FIG. 3 illustrates a illustrates an exploded isometric view of the gas/liquid separation module shown in FIGS. 1A-2D, according to some embodiments.

FIG. 4 illustrates a diagrammatic view of an exemplary aseptic sampling device including multiple bioreactors, according to some embodiments.

FIG. 5 illustrates a diagrammatic view of an exemplary aseptic sampling device including a gas/liquid separator inside of the aseptic zone, according to some embodiments.

FIG. 6 illustrates a diagrammatic view of an exemplary aseptic sampling device including multiple reservoirs and a single gas/liquid separator, according to some embodiments.

FIG. 7 illustrates a diagrammatic view of an exemplary aseptic sampling system including a cleaning agent reservoir configured to sterilize a portion of the intermediate flow path, according to some embodiments.

FIG. 8A illustrates a diagrammatic view of an exemplary aseptic sampling system including a selection valve located between the reservoir and the gas/liquid separator, according to some embodiments.

FIG. 8B illustrates a diagrammatic view of an exemplary aseptic sampling system including a selection valve located between the reservoir and the gas/liquid separator, according to some embodiments.

FIG. 8C illustrates a diagrammatic view of an exemplary aseptic sampling system including a selection valve located between the reservoir and the gas/liquid separator, according to some embodiments.

FIG. 9A illustrates a diagrammatic view of an exemplary aseptic sampling system including a selection valve located between the reservoir and the gas/liquid separator, and an injection valve, according to some embodiments.

FIG. 9B illustrates a diagrammatic view of an exemplary aseptic sampling system including a selection valve located between the reservoir and the gas/liquid separator, and an injection valve, according to some embodiments.

FIG. 9C illustrates a diagrammatic view of an exemplary aseptic sampling system including a selection valve located between the reservoir and the gas/liquid separator, and an injection valve, according to some embodiments.

FIG. 10 illustrates an exemplary flow chart of a method for sampling a liquidous material with a sampling system, according to some embodiments.

FIG. 11 illustrates an exemplary flow chart of a method of sampling a liquidous material, according to some embodiments.

FIG. 12A illustrates a diagrammatic view of an exemplary aseptic sampling system including multiple reservoirs and a pinch valve, according to some embodiments.

FIG. 12B illustrates a diagrammatic view of an exemplary aseptic sampling system including multiple reservoirs and a pinch valve, according to some embodiments.

FIG. 12C illustrates a diagrammatic view of an exemplary aseptic sampling system including multiple reservoirs and a pinch valve, according to some embodiments.

FIG. 13A illustrates a diagrammatic view of an exemplary aseptic sampling system including a peristaltic pump, according to some embodiments.

FIG. 13B illustrates a diagrammatic view of an exemplary aseptic sampling system including a peristaltic pump, according to some embodiments.

FIG. 13C illustrates a diagrammatic view of an exemplary aseptic sampling system including a peristaltic pump, according to some embodiments.

### DETAILED DESCRIPTION

The devices, systems, and methods disclosed herein are directed toward a sampling system configured to accurately control the amount of sample sent to an analyzer and minimize sample loss. The system is an aseptic sampling system, meaning the sample is delivered to the analyzer through a sterile process, i.e., without contaminants or cross-contaminates. The aseptic sampling system may include a gas/liquid separator configured to remove a gas from liquid sample material. The gas/liquid separator may include a separation chamber including a gas-permeable, liquid impermeable membrane. The membrane may divide the separation chamber into a permeate portion and a retentate portion. The separation chamber may be fluidically connected to a pressure regulation system configured to control a gas pressure in the permeate portion. The pressure regulation system may be in pneumatic connection with the retentate portion of the separation chamber, i.e., a pressure change caused by the pressure regulation system may permeate through the membrane and into the retentate portion. The pressure regulation system may be configured to pull liquid material through a flow path (i.e., a channel and/or tubing to direct fluid flow) in a direction toward the separation chamber and/or to push liquid material through the flow path (in a direction away from the separation chamber). Pushing and/or pulling the liquid sample material via the gas/liquid separator is beneficial, as it enables the sample to be primed at a first stage without requiring the flow path to be connected to a pump. The gas/liquid separator may also enable rapid cleaning or clearing of the flow path, and minimize sample loss, as liquid material may be pushed out from the flow path back into a reservoir.

The aseptic sampling system may be automated, meaning that a control unit and/or processor may be configured to control various functions of the system, with or without concurrent user input. For instance, a set of instructions may be stored in the control unit and the control unit may output the instructions to the aseptic sampling system (e.g., actuation of a valve, movement of a pump, control a gas pressure, etc.). In some embodiments, the aseptic sampling system may include a user interface for a user to control function(s) and/or issue instructions to the aseptic sampling system.

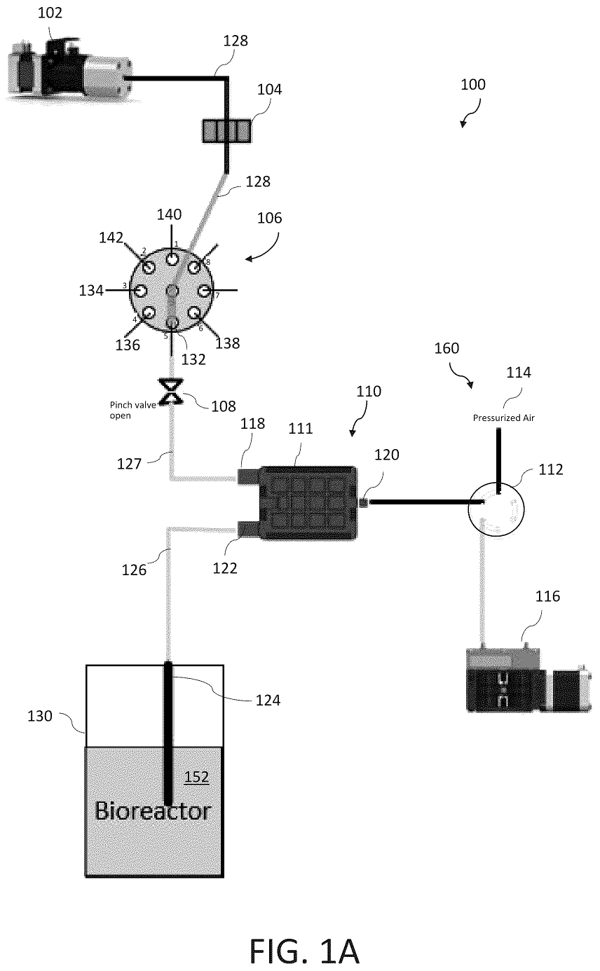

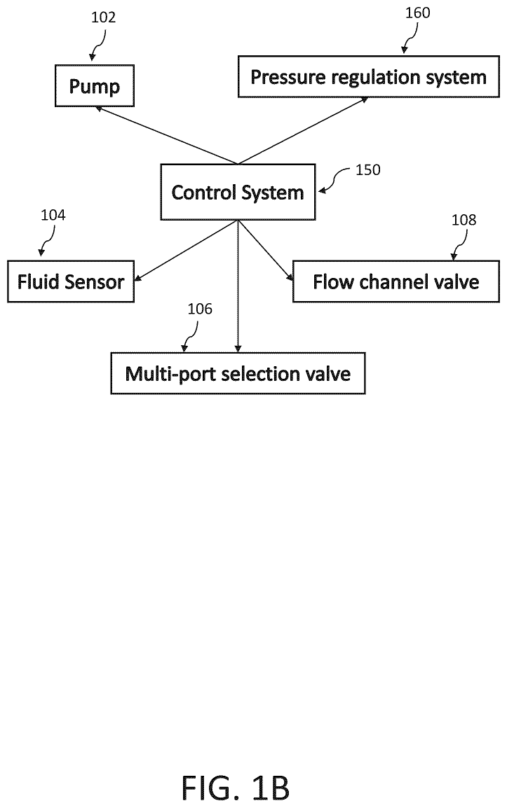

FIG. 1A illustrates a diagrammatic view of an aseptic sampling system 100, according to some embodiments. The aseptic sampling system 100 includes a pump 102, a multi-port selection valve 106, a gas/liquid separator 110, and a probe 124. The probe 124 may be configured to extend into a reservoir 130 (e.g., a bioreactor, cell culture, or tank) to extract a liquid material 152 (i.e., a sample). The probe 124 may be fluidically connected to the gas/liquid separator 110 through a first flow path 126. In some embodiments, the first flow path 126 is an elongate tube or line. The first flow path 126 may be secured to the gas/liquid separator 110 at a first port 122. The gas/liquid separator 110 includes a gas/liquid separation module 111 and a pressure regulation system 160.

As described hereinafter, the gas/liquid separator 110 may include any of the features or elements described in commonly owned, U.S. Pat. No. 9,381,449 filed on Jun. 6, 2013 and entitled “Carbon Nanotube Composite Membrane,” U.S. Pat. No. 9,370,734 filed on Jun. 13, 2014 and entitled “Fluid Degassing Module with Helical Membrane,” U.S. Pat. No. 9,656,186 filed on May 29, 2015 and entitled “Fluid Degassing Apparatus,” U.S. Pat. No. 9,962,661 filed on Jun. 30, 2016 and entitled “Composite Membrane,” U.S. Pat. No. 10,143,942 filed on Aug. 29, 2016 and entitled “Membrane Gas/Liquid Contactor,” and U.S. Pat. No. 10,953,348 filed on Nov. 30, 2018 and entitled “Membrane Gas/Liquid Contactor,” the contents of which are hereby incorporated by reference in their entirety.

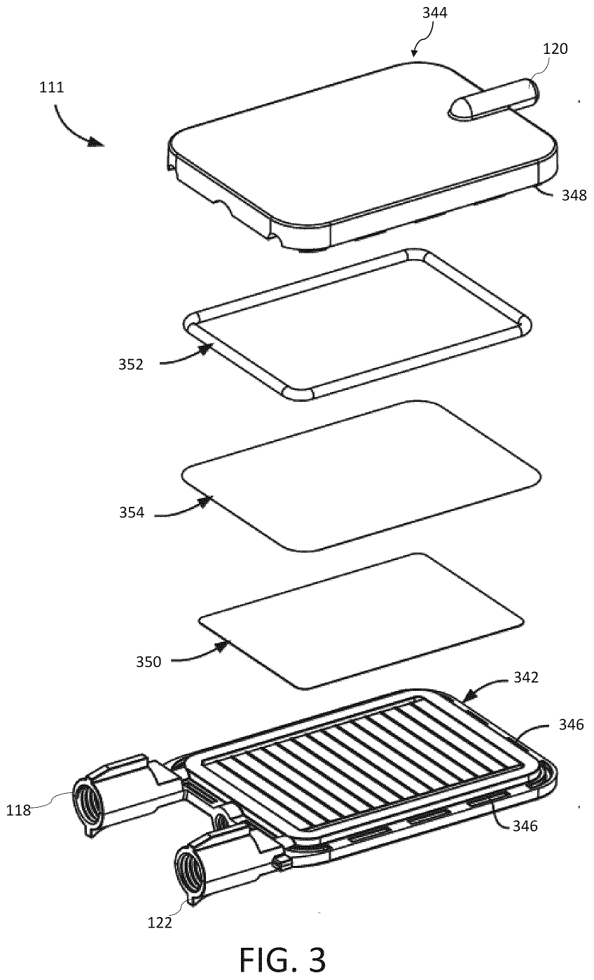

The gas/liquid separator 110 includes the gas/liquid separation module 111 (further illustrated in FIG. 3) having a separation chamber and a membrane 350. The membrane 350 may be gas-permeable (i.e., allowing gas and pneumatic pressure to permeate through the membrane) and may be liquid-impermeable. The membrane may divide the separation chamber into a retentate portion (also referred to as the retentate side) and a permeate portion (also referred to as the permeate side). The retentate portion may be directly fluidically connected to the first flow path 126, such as not through the membrane 350. Thus, the retentate portion may be configured to receive the liquid material 152 therein. The permeate portion may be directly fluidically connected to the pressure regulation system 160 of the gas/liquid separator 110, in some embodiments not through membrane 350. The pressure regulation system 160 may include a pressurized air source 114, a vacuum source 116, and a pressure regulation control valve 112. In some embodiments, the pressure regulation system 160 may be operated between a first mode to establish a first gas pressure condition in the permeate portion of the separation chamber and a second mode to establish a second gas pressure condition.

The first mode may include fluidically connecting the vacuum source 116 to the gas/liquid separation module 111 via the pressure regulation control valve 112, and operating the vacuum source 116 to create a low pressure condition (also referred to as the first gas pressure condition and/or negative gas pressure) in the permeate portion of the separation chamber. The low pressure condition in the permeate portion may create a low pressure condition in the retentate portion of the gas/liquid separation module 111. The establishment of the low pressure condition in the retentate portion of the gas/liquid separation module 111 is facilitated by a gaseous environment, such as air, at the retentate portion, which may be evacuated through the membrane 350 to leave the low pressure condition. The low pressure condition may provide a suction force to the first flow path 126 and the probe 124. Thus, the liquid material 152 may be pulled from the reservoir 130 and/or probe 124 to the gas/liquid separation module 111 via the low pressure condition of the pressure regulation system 160. In other words, the pressure regulation system 160 may be in pneumatic communication with the first flow path 126 through the gas/liquid separation module 111 and the membrane 350 of the gas/liquid separator 110. The first mode may be configured to degas the liquid material 152, i.e., to remove gas or gas bubbles from the liquid material 152 in the retentate portion of the gas/liquid separation module 111 through the gas-permeable membrane 350. The gas and/or gas bubbles permeates through the membrane 350 to the permeate portion of the separation chamber. In some embodiments, the first flow path 126 may be cleared prior to the first mode by filling the first flow path 126 with gas or air, as for instance, if the first flow path 126 is filled with liquid material, the retentate portion of the gas/liquid separation module 111 may fill prematurely and prevent liquid material from being pulled from the reservoir 130.

The second mode may include fluidically connecting the pressurized air source 114 to the gas/liquid separation module 111 via the pressure regulation control valve 112, and operating the pressurized air source 114 to create a high pressure condition (also referred to as the second gas pressure condition and/or positive gas pressure) in the permeate portion of the separation chamber. The high pressure condition in the permeate portion may permeate through the membrane 350 and create a high pressure condition in the retentate portion of the gas/liquid separation module 111. The high pressure condition permeating through the membrane 350 may provide a push force to the retentate portion of the gas/liquid separation module 111 and to the first flow path 126. Thus, any liquid material 152 or gas disposed between the retentate portion of the gas/liquid separation module 111 and the probe 124 may be pushed out of the first flow path 126, and, in some embodiments, toward and/or into the reservoir 130. In some embodiments, the liquid material 152 pushed out of the first flow path 126 toward the reservoir may prevent solid accumulation in the probe 124 and/or first flow path 126. For instance, in some embodiments, the probe 124 may include a cell-free sampling filter with a micro-porous membrane, and in some cases, placing the probe 124 in the reservoir 130 may result in the accumulation of solid materials (e.g., cell, cell debris, and/or other solids) in the filter and block the filter pores. The pushback of liquid material 152 out of the probe 124 may dislodge solid materials from the filter in a form of “backflushing”, and thereby prevent solid accumulation.

Another benefit of the second mode is providing a motivating pressure source that is free from particles or microorganisms. Conventional sources of pressurized gas/air for fluid motivation in fluid sampling systems often are filtered in an attempt to achieve an aseptic environment. However, the best filters typically have pore sizes in the range of 0.2-5 μm, which are large enough to allow the undesired passage of certain microorganisms and other particles. The separation membranes of the present invention may be non-porous, which prevent passage of particles and microorganisms. Thus, in the case of the second mode of the pressure regulation system, fully pure gas may be employed as a motivating force for fluid flow through fluid paths of the aseptic sampling system 100.

The gas/liquid separator 110 may be fluidically connected to the multi-port selection valve 106. A degassed liquid material 152 may exit the retentate portion of the gas/liquid separation module 111 through a second port 118. An intermediate flow path 127 may fluidically connect the gas/liquid separator 110 to the multi-port selection valve 106. In some embodiments, the intermediate flow path 127 may include a flow path valve 108 (i.e., a pinch valve) configured to selectively open and close the intermediate flow path 127. The flow path valve 108 may be configured to hold the liquid material 152 in a primed position in the intermediate flow path 127. In some embodiments, the flow path valve 108 may be operated to open only when a sample port 132 is fluidically connected to the pump 102 during pump aspiration, and to otherwise be closed. In some embodiments, the flow path valve 108 is an aseptic barrier. A second flow path 128 located between the multi-port selection valve 106 and the pump 102 may be sterilizable via a cleaning agent through port 138 or other sterilization process, and thus, is “aseptic” or substantially free from biological contaminants.

The multi-port selection valve 106 may be fluidically connected to the pump 102. The pump 102 may include a displacement pump, a piston pump, a syringe pump, a peristaltic pump, or any other useful sampling pump. In some embodiments, the pump 102 may be a displacement pump to ensure accurate sampling amounts are sampled from the reservoir. The pump 102 may be fluidically connected to the multi-port selection valve 106 via the second flow path 128. In some embodiments, the second flow path 128 may include a fluid sensor 104 (e.g., a bubble sensor) configured to detect a fluid or gas passing through the second flow path 128. The fluid sensor 104 may be configured to detect the presence and/or absence of air bubbles in the liquid material 152. The pump 102 may be configured to aspirate (i.e., pull air or fluid toward the pump) and expirate (i.e., push away air or fluid from the pump).

The multi-port selection valve 106 may include a plurality of ports, including but not limited to the sample port 132, a waste port 134, an analyzer port 136, a cleaning agent port 138, a working fluid port 140 and/or an air port 142. The multi-port selection valve 106 may selectively fluidically connect the pump 102 to the plurality of ports (e.g., 132, 134, 136, 138, 140, 142, etc.). For example, FIG. 1A illustrates the multi-port selection valve 106 fluidically connecting the sample port 132 to the pump 102. The sample port 132 is in fluidic connection with the intermediate flow path 127, and thus, the sample port 132 fluidically connects the gas/liquid separator 110, the first flow path 126, and the probe 124 to the pump 102. In other words, a sample of liquid material 152 can be drawn from the reservoir 130 to the pump 102 through the sample port 132.

The pump 102 may be configured to aspirate gas or liquid through a first port of the multi-port selection valve 106 into the second flow path 128, and expirate the gas or liquid through a second port. For instance, the pump 102 may be fluidically connected to the sample port 132, and the liquid material 152 may be aspirated into the second flow path 128. The multi-port selection valve 106 may be actuated to fluidically connect the pump 102 to the analyzer port 136. The pump 102 may then expirate the liquid material 152 through the analyzer port 136 to a downstream analyzer. In some embodiments, the multi-port selection valve 106 may be configured to enable system 100 to dilute, catalyze, or otherwise modify the extracted liquid material 152. For instance, the pump 102 may be fluidically connected to the sample port 132, such that a liquid material 152 may be aspirated by pump 102 into the second flow path 128. The multi-port selection valve 106 may be actuated to fluidically connect the pump 102 to the working fluid port 140, and the pump 102 may further aspirate a working fluid (e.g., a catalyst, enzyme, buffer, calibrant, or other active material) into the second flow path 128. In some embodiments, the multi-port selection valve 106 may include a port that may be fluidically connected to a downstream analyzer (e.g., a HaLCon™ protein analyzer). In some embodiments, the multi-port selection valve 106 may include a port that may be fluidically connected to a transport vessel (e.g., a test tube) to analyze the sample with an unconnected analyzer.

The plurality of ports illustrated in FIG. 1A are exemplary embodiments. Various ports may be included in the multi-port selection valve 106 and selectively connected to the pump 102. For example, various cleaning agents, various working fluids, various analyzers, additives, calibrants, buffers, and/or exit ports may be selectively connected to the pump 102 via the multi-port selection valve 106.

In some embodiments, the aseptic sampling system 100 may include a control system 150 (see FIG. 1B) communicatively connected to the pump 102, the fluid sensor 104, the multi-port selection valve 106, the flow path valve 108, and/or the pressure regulation system 160 (including the pressurized air source 114, the vacuum source 116, and the pressure regulation control valve 112). The control system 150 may be configured to control and/or receive data from the components described above. For example, the control system may be configured to selectively open, close, or actuate one or more valves, selectively control pressure in the gas/liquid separation module 111 and/or flow paths, and selectively aspirate or expirate the pump 102. The control system 150 may be programmed to carry out a set or commands or instructions and may be configured to receive inputs from users to selectively control one or more functions of the aseptic sampling system 100. The control system 150 may be configured to control one or more elements of the pressure regulation system 160 to select between the first mode (low pressure condition) and the second mode (high pressure condition) in the gas/liquid separation module 111.

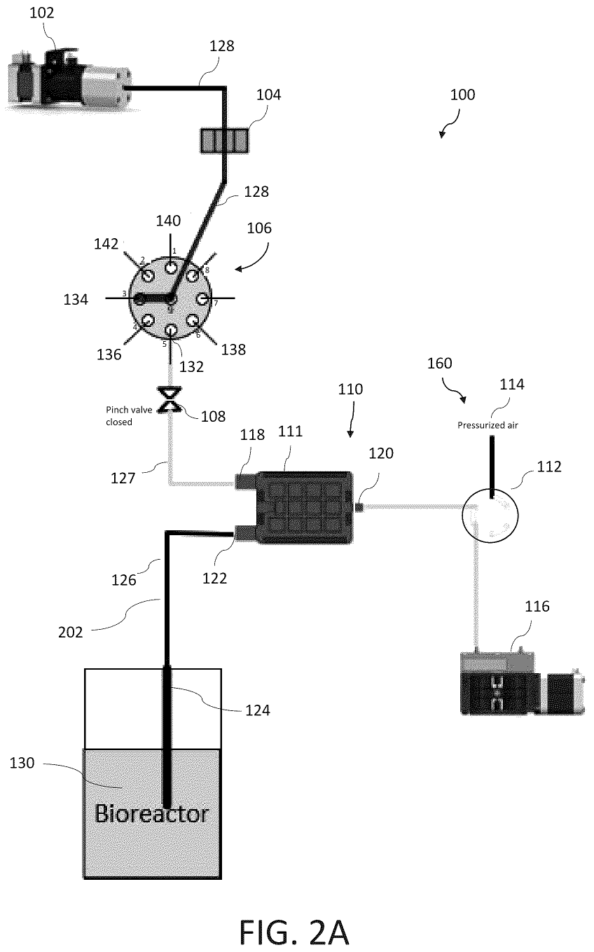

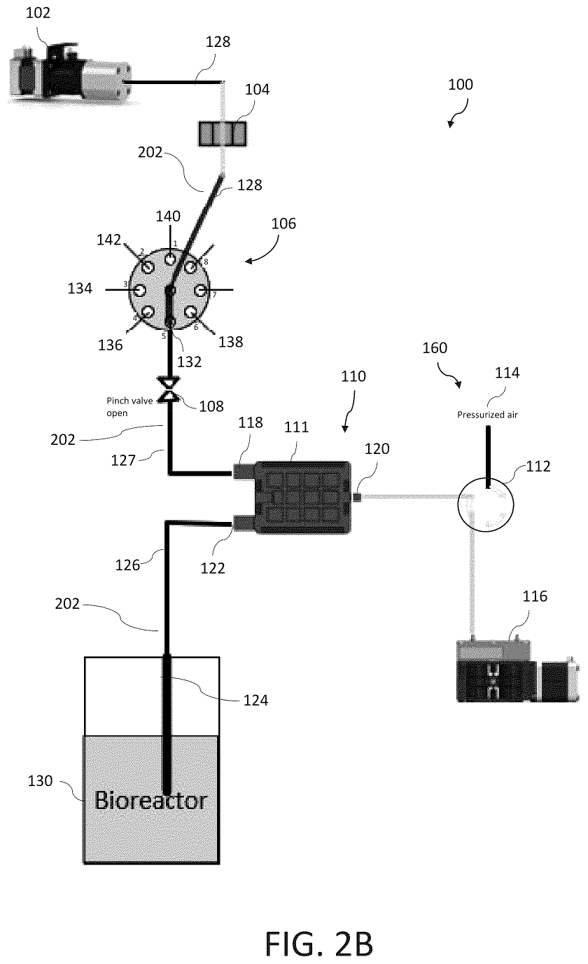

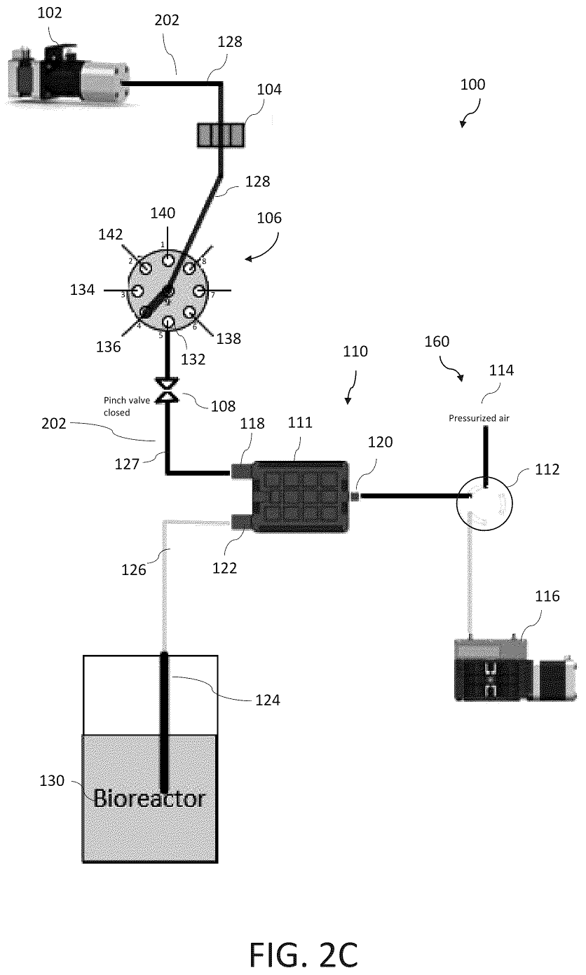

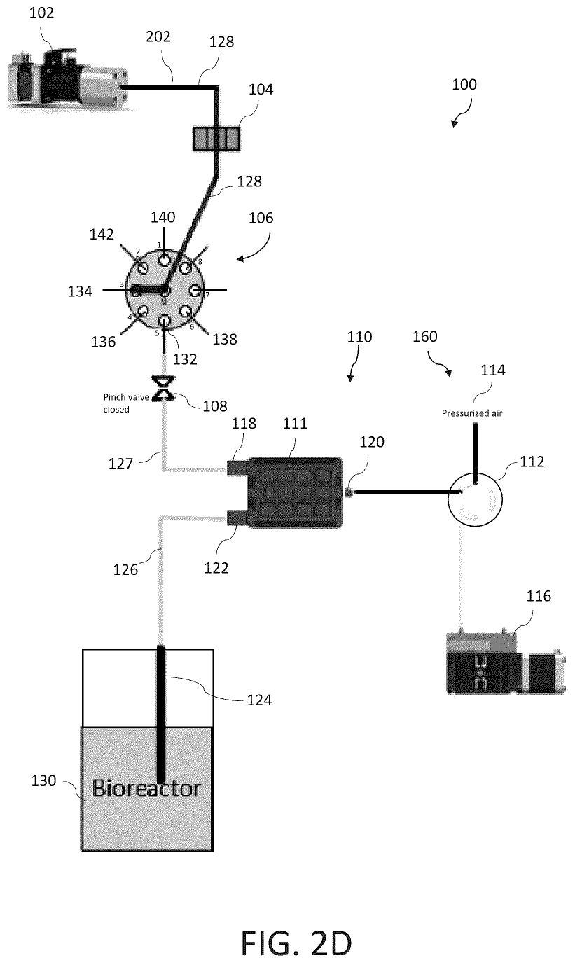

FIGS. 2A-D illustrate an exemplary progression of automated aseptic sampling, according to some embodiments. FIG. 2A illustrates a diagrammatic view of the aseptic sampling system 100 in a first step of sampling. In some embodiments, the aseptic sampling system 100 may be cleared before the first step illustrated in FIG. 2A, including, for example, filling or ensuring that the first flow path 126, the gas/liquid separation module 111, and the intermediate flow path 127 are filled with air or gas to eliminate residue from a prior procedure. The first step may include selectively connecting the vacuum source 116 to the gas/liquid separation module 111 via the pressure regulation control valve 112. The vacuum source 116 may create a low gas pressure condition in the permeate portion of the gas/liquid separation module 111. The low gas pressure condition may permeate through the membrane 350 and act as a suction force to draw a sample 202 from the reservoir 130 to the gas/liquid separator 110. As illustrated in FIG. 2A, the sample port 132 is not connected to the pump 102 during this initial operation. The only force acting upon the sample 202 to draw the sample 202 from the reservoir 130 to the gas/liquid separator 110 is the low pressure condition created by the pressure regulation system 160. The gas/liquid separator 110 may be operated in a manner to remove gas (e.g., air bubbles) from the sample 202. The flow path valve 108 disposed on the intermediate flow path 127 is closed in the condition illustrated in FIG. 2A.

FIG. 2B illustrates a diagrammatic view of the aseptic sampling system 100 in a second step of sampling, according to some embodiments. The sample port 132 of selection valve 106 is fluidically connected to the pump 102, and the pump aspirates the sample 202 from the gas/liquid separation module 111 through the intermediate flow path 127 and multi-port selection valve 106, to the second flow path 128. The vacuum source 116 is fluidically connected to the permeate portion of the gas/liquid separation module 111, creating a low pressure condition to remove gas, such as air bubbles, through the gas-permeable membrane 350. The sample drawn through the gas/liquid separator is preferably degassed (i.e., bubble free), and therefore, an accurate sample volume can be aspirate/dispensed. Accurate sample volume delivery is beneficial when used in analytical characterization, such as titer measurement. The flow path valve 108 disposed on the intermediate flow path 127 is open during this second step.

FIG. 2C illustrates a diagrammatic view of the aseptic sampling system 100 in a third step of sampling, according to some embodiments. The sample 202 may be located in the second flow path 128 between the pump 102 and the multi-port selection valve 106. The flow path valve 108 disposed on the intermediate flow path 127 is closed in this third step to keep the system aseptic. The multi-port selection valve 106 is actuated to fluidically connect the pump 102 to an analyzer (not shown) via the analyzer port 136. The pump 102 expirates to dispense the sample 202 through the analyzer port 136 towards a downstream analyzer. The pressure regulation control valve 112 is actuated to fluidically connect the gas/liquid separator 110 to the pressurized air source 114 of the pressure regulation system 160. The pressurized air source 114 creates a high gas pressure condition in the permeate portion of the gas/liquid separation module 111 which permeates through the membrane 350 to force the sample 202 from the gas/liquid separation module 111 through the first flow path 126 toward the reservoir 130. As shown in FIG. 2C, the first flow path 126 does not contain any of the sample 202, as the sample 202 has been forced out of the gas/liquid separation module 111 and into the reservoir 130. This helps minimize sample loss, as the sample 202 disposed between the gas/liquid separator 110 and the reservoir 130 is not ejected to waste, but instead, dispensed back into the reservoir 130. The sample 202 disposed between the gas/liquid separator 110 and the multi-port selection valve 106 in the intermediate flow path 127 remains. In some embodiments, the intermediate flow path 127 may be minimized (i.e., shortened) in order to minimize sample loss.

FIG. 2D illustrates a diagrammatic view of the aseptic sampling system 100 in a fourth step of sampling, according to some embodiments. The remaining sample 202 disposed in the intermediate flow path 127 as shown in FIG. 2C is aspirated into the second flow path 128 via actuation of the multi-port selection valve 106 to fluidically connect the pump 102 to the sample port 132 with the flow path valve 108 open. Once all the remaining sample 202 is aspirated from the intermediate flow path 127 to the path flow path 128 and the flow path valve 108 closes, the multi-port selection valve 106 is then actuated to fluidically connect the pump 102 to the waste port 134. The pump 102 may then be operated to expirate the sample 202 through the waste port 134 to a waste container (not shown). The clean air from the gas/liquid separator 110 continues to purge through the probe 124 and keep the probe 124 unblocked.

FIG. 3 illustrates an exploded isometric view of the gas/liquid separation module 111 shown in FIGS. 1A-2D, according to some embodiments. The gas/liquid separation module 111 includes a first plate 342 and a second plate 344, the first plate 342 and the second plate 344 fitting together to form the separation chamber for the gas/liquid separation module 111. In some embodiments, the first plate 342 may include one or more mounting receptacles 346 configured to receive one or more mounting tabs 348 located on the second plate 344. The gas/liquid separation module may include a gasket 352, a diffuser element 354, and a membrane 350.

The first plate 342 may define a fluid flow path for contact between the fluid flow and the membrane 350. Or in other words, the retentate portion of the gas/liquid separation module 111 may be disposed between the first plate 342 and the membrane 350. The second plate 344 may include a pressure regulation port 120 configured to fluidically connect the pressure regulation system 160 to the gas/liquid separation module 111. The permeate portion of the gas/liquid separation module 111 may be disposed between the second plate 344 and the membrane 350. The membrane 350 may be gas-permeable to allow gas to permeate through the membrane 350. In some embodiments, membrane 350 may be gas-permeable but non-porous. Therefore, if a low pressure or low partial pressure condition is present in the permeate portion of the gas/liquid separation module 111, gas will be drawn from the retentate portion to the permeate portion through the membrane 350. The membrane 350 may be impermeable to the liquid material 152, i.e., the liquid material 152 cannot pass through the membrane 350.

It should be noted that the gas/liquid separation module 111 illustrated in FIG. 3 is merely one embodiment. The gas/liquid separator 110 used in the aseptic sampling system may include any of the features or elements described in commonly owned, U.S. Pat. No. 9,381,449 filed on Jun. 6, 2013 and entitled “Carbon Nanotube Composite Membrane,” U.S. Pat. No. 9,370,734 filed on Jun. 13, 2014 and entitled “Fluid Degassing Module with Helical Membrane,” U.S. Pat. No. 9,656,186 filed on May 29, 2015 and entitled “Fluid Degassing Apparatus,” U.S. Pat. No. 9,962,661 filed on Jun. 30, 2016 and entitled “Composite Membrane,” U.S. Pat. No. 10,143,942 filed on Aug. 29, 2016 and entitled “Membrane Gas/Liquid Contactor,” and U.S. Pat. No. 10,953,348 filed on Nov. 30, 2018 and entitled “Membrane Gas/Liquid Contactor,” the contents of which are hereby incorporated by reference in their entirety. Moreover, it is to be understood that various embodiments for gas/liquid separator 110 are contemplated as being useful in the system of the present invention. An example alternative type of gas/liquid separator useful in the present invention is a hollow fiber silicone rubber style degasser that is known in the art.

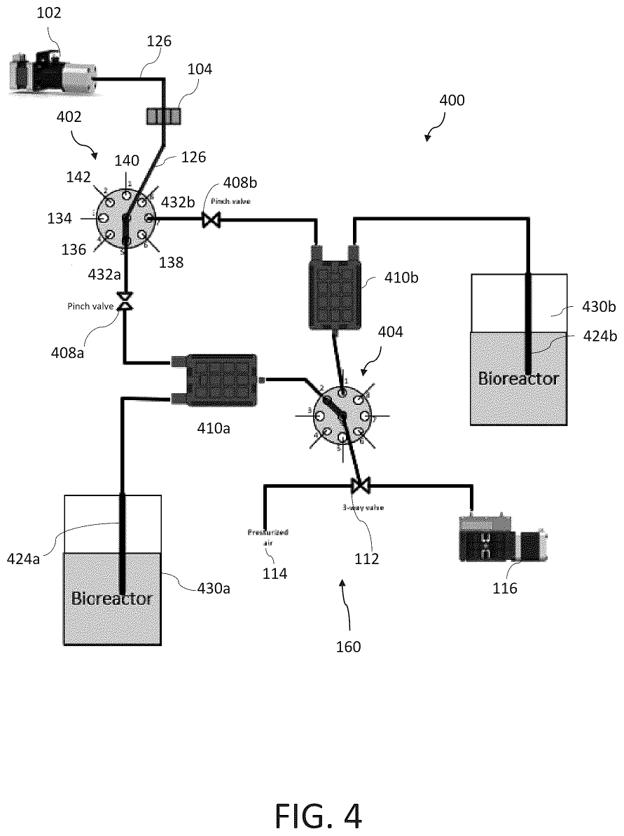

FIG. 4 illustrates a diagrammatic view of an exemplary aseptic sampling system 400 including a plurality of reservoirs. A single pressure regulation system 160 may be used with a first reservoir 430a and a second reservoir 430b. The pressure regulation system 160 may be fluidically connected to the first reservoir 430a and/or the second reservoir 430b via a reservoir selection valve 404. The first reservoir 430a may be fluidically connected to a first probe 424a, a first gas/liquid separator 410a, and a first flow path valve 408a. The second reservoir 430b may be fluidically connected to a second probe 424b, a second gas/liquid separator 410b, and a second flow path valve 408b. The first reservoir 430a and the second reservoir 430b may be selectively fluidically connected with the pump 102 via a multi-port selection valve 402. The multi-port selection valve 402 may include a first sample port 432a in fluidic connection with the first reservoir 430a, and a second sample port 432b in fluidic connection with the second reservoir 430b. The valve 404 may be selectively operated to fluidically connect with different bioreactors and provide clean air/gas to flush the probes in the bioreactor to prevent probe blockage.

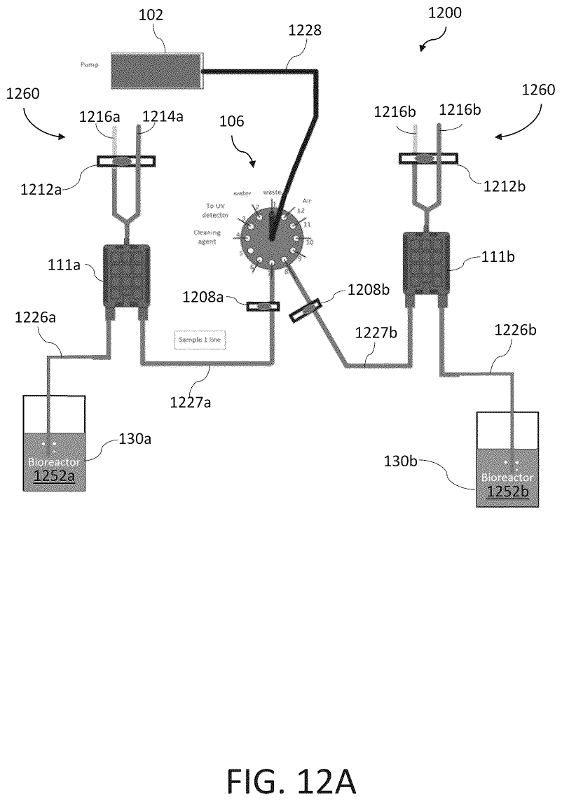

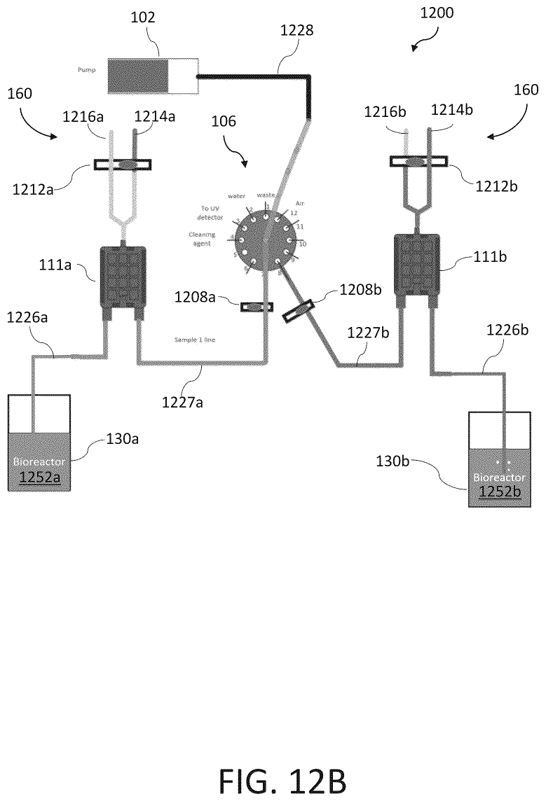

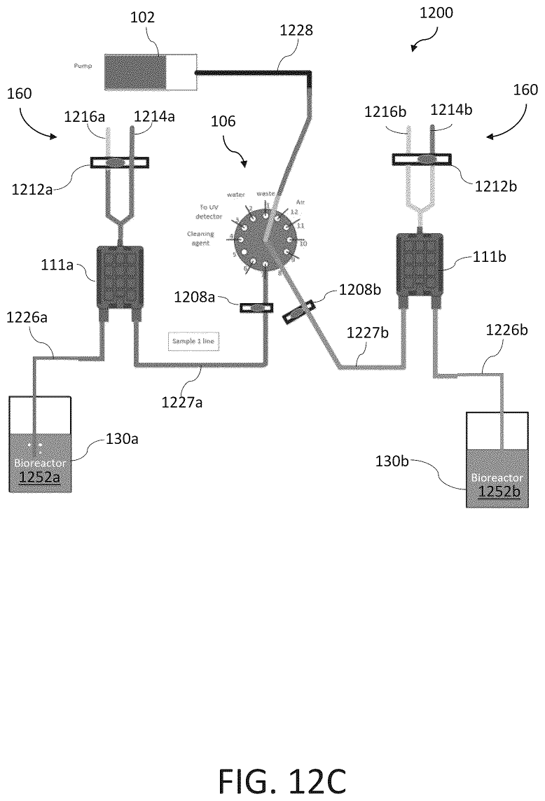

FIGS. 12A-C illustrates a diagrammatic view of an aseptic sampling system 1200 including one or more reservoirs 130a, 130b and a pump 102, according to some embodiments. The aseptic sampling system 1200 may include first flow paths 1226a, 1226b fluidically connecting the reservoirs 130a, 130b to respective gas/liquid separation modules 111a, 111b. The gas/liquid separation modules 111a, 111b may be fluidically connected to a pressure regulation system 1260. The pressure regulation system 1260 may include a vacuum source that is capable of providing a negative pressure to the permeate side of the gas/liquid separation modules 111a, 111b though vacuum lines 1216a, 1216b, and an gas source delivering or providing access to gas, such as air, at the permeate side of the gas/liquid separation module 111 through gas lines 1214a, 1214b. In some embodiments, the gas source may deliver a positively pressurized gas, whereas in other embodiments, the gas source may provide access to a neutral gas (i.e., air having roughly 1 atm of pressure).

The pressure regulation system 1260 may include pressure regulation control valves 1212a, 1212b to control the fluidic connection of the gas source and the vacuum source to the respective gas/liquid separation modules 111a, 111b. In some embodiments, the pressure regulation control valves 1212a, 1212b may be binary valves having two states, with the first state fluidically connecting the air source to the respective gas/liquid separation modules 111a, 111b through respective gas lines 1214a, 1214b, and the second state fluidically connecting the vacuum source to the respective gas/liquid separation modules 111a, 111b through vacuum lines 1216a, 1216b. For instance, FIG. 12A illustrates the pressure regulation control valves 1212a, 1212b in the first state wherein the vacuum source is fluidically disconnected from the gas/liquid separation modules 111a, 111b and the gas source fluidically connected to the gas/liquid separation modules 111a, 111b via gas lines 1214a, 1214b. FIG. 12B illustrates the first pressure regulation control valve 1212a in the second state, wherein the gas source is fluidically disconnected from the first gas/liquid separation module 111a, and the vacuum source being fluidically connected to the first gas/liquid separation module 111a via vacuum line 1216a. In this state, fluid from reservoir 130a may be drawn along flow path 1226a by a negative pressure established in gas/liquid separation module 111a through a separation membrane in module 111a. In some embodiments, actuation of the pressure regulation control valve 1212a fluidically connects one path (i.e., the air source or the vacuum source) while simultaneously disconnecting the other path.

The aseptic sampling system 1200 may include intermediate flow paths 1227a, 1227b fluidically connecting the gas/liquid separation modules 111a, 111b to the multi-port selection valve 106. The intermediate flow paths 1227a, 1227b may each include respective flow path valves 1208a, 1208b. The flow path valves 1208a, 1208b may be pinch valves configured to selectively close off the respective intermediate flow path 1227a, 1227b. The multi-port selection valve 106 may include any and/or all of the ports described above with respect to FIGS. 1A-4 to fluidically connect the intermediate flow paths 1227a, 1227b and/or secondary flow path 1228 to a plurality of flow paths, reservoirs, downstream analyzers, waste ports, etc.

The aseptic sampling system 1200 may include the secondary flow path 1228 fluidically connecting the multi-port selection valve 106 to one or more pumps 102. The pump 102 may be a displacement pump including a piston configured to recede within a cavity (i.e., drawing fluid toward the pump) and/or configured to press forward within the cavity (i.e., pushing fluid away from the pump). Stated differently, the pump 102 may provide bidirectional movement of the sample 1252a, 1252b through the combined flow path-a first direction drawing fluid toward the pump 102 and a second direction pushing fluid away from the pump 102. The secondary flow path 1228 may be configured to store fluid downstream from the multi-port selection valve 106. The multi-port selection valve 106 may be actuated with the fluid stored within the secondary flow path 1228 to selectively fluidically connect another valve port to the secondary flow path 1228.

According to some embodiments, a process of aseptic sampling of a sample 1252a with the aseptic sampling system 1200 is described below. The process may begin with the first pressure regulation valve 1212a in the first state (i.e., the vacuum source fluidically disconnected from the first gas/liquid separation module 111a), the first flow path valve 1208a closing off the intermediate flow path 1227a, and the multi-port selection valve 106 disconnected from the intermediate flow path 1227a.

The pressure regulation valve 1212a may be actuated to fluidically connect the vacuum source to the first gas/liquid separation module 111a. Negative pressure from the vacuum source may permeate through the membrane 350 of the first gas/liquid separation module 111a, and may accordingly provide a suction force in the first flow path 1226a. The suction force in the first flow path 1226a may draw the sample 1252a into the first flow path 1226a and into the retentate portion of the first gas/liquid separation module 111a.

When the retentate portion of the first gas/liquid separation module 111a is at least partially filled, the multi-port selection valve 106 may be actuated to fluidically connect the secondary flow path 1228 to the intermediate flow path 1227a. The first flow path valve 1208a may be actuated to open the intermediate flow path 1227a. The pump 102 may generate a suction force within the flow path (i.e., with the piston receding within the cavity), and thus, the sample 1252a may be drawn from the first gas/liquid separation module 111a to the intermediate flow path 1227a and into the secondary flow path 1228 through selection valve 106.

In some embodiments, the process may include an accurate sampling step, wherein the first flow path valve 1208a is closed and the multi-port selection valve 106 is actuated to fluidically connect the secondary flow path 1228 to a waste port. The pump 102 may generate a positive pressure within the flow path (i.e., with the piston extending) and push any sample located within the secondary flow path 1228 out through the waste port. The amount of sample 1252a can be accurately controlled, as the entire intermediate flow path 1227a may be filled with the sample 1252a and none of the secondary flow path 1228 is filled with the sample 1252a. Any pumping of the intermediate flow path 1227a will immediately fill the secondary flow path 1228, and thus, the amount of sample 1252a drawn into the secondary flow path 1228 can be accurately measured/controlled via the pump 102.

The multi-port selection valve 106 may actuate to fluidically connect the intermediate flow path 1227a to the secondary flow path 1228 and the flow path valve 1208a may be opened. The pump 102 may generate a negative pressure within the combined flow path to urge the sample 1252a into the secondary flow path 1228. After a controlled amount of sample 1252a is drawn into the secondary flow path 1228, the flow path valve 1208 may be closed and the selection valve may be actuated to fluidically connect the secondary flow path 1228 to a downstream analyzer port. The pump 102 may generate a positive pressure within the combined flow path to urge the sample 1252a to a downstream analyzer.

In some embodiment, the process may include a clearing step. The pressure regulation valve 1212 may be actuated to fluidically connect the air source 1214 to the gas/liquid separation module 111. The air source 1214 may urge the sample 1252a located within the first flow path 1226a back toward the reservoir 130. The flow path valve 1208a may actuate to open the intermediate flow path 1227a and the multi-port selection valve 106 may actuate to fluidically connect the intermediate flow path 1227a to the secondary flow path 1228. The remaining sample 1252a may be drawn from the intermediate flow path 1227a to the secondary flow path 1228 via the pump. The multi-port selection valve 106 may actuate to fluidically connect the secondary flow path 1228 to a waste port, and the remaining sample 1252a may be disposed through the waste port.

In some embodiments, the above process may be repeated for the second bioreactor (i.e., reservoir 130b connected to the first flow path 1226b and the intermediate flow path 1227b). In some embodiments, the second bioreactor may be prepared while the above process is occurring. For example, FIG. 12C illustrates the aseptic sampling system 1200 with the second bioreactor primed for aseptic sampling of the sample 1252b.

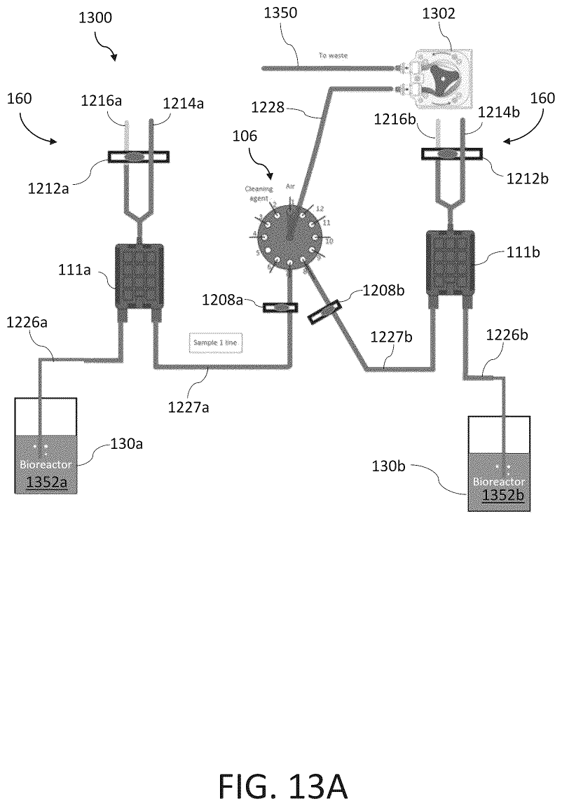

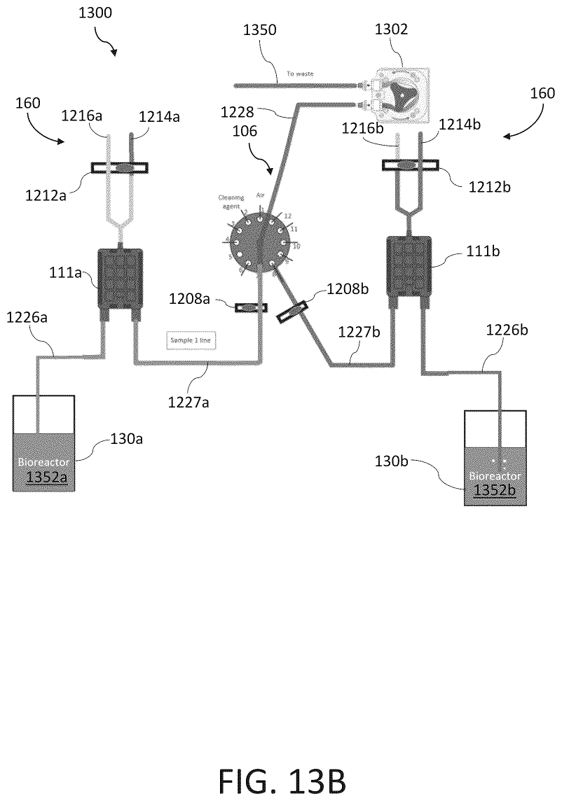

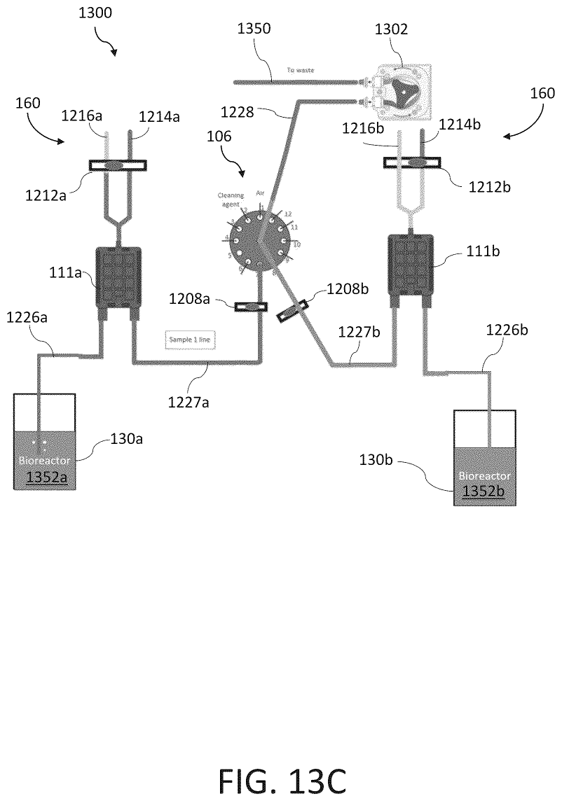

FIGS. 13A-C illustrates a diagrammatic view of an aseptic sampling system 1300 including a peristaltic pump 1302, according to some embodiments. The aseptic sampling system 1300 may include any or all of the features described above in the aseptic sampling system 1200, albeit with the peristaltic pump 1302 as opposed to the one or more pumps 102 in the aseptic sampling system 1200. The peristaltic pump 1302 may be unidirectional, i.e., can only move fluid in one direction along the combined flow path. For example, the peristaltic pump 1302 may move fluid from the multi-port selection valve 106 to the secondary flow path 1228, through the peristaltic pump 1302, and through a downstream flow path 1350.

According to some embodiments, a process of aseptic sampling of a sample 1352a with the aseptic sampling system 1300 is described below. The process may begin with the pressure regulation valve 1212a in the first state (i.e., the vacuum source fluidically disconnected from the first gas/liquid separation module 111a), the flow path valve 1208a closing off the intermediate flow path 1227a, and the multi-port selection valve 106 disconnected from the intermediate flow path 1227a.

The pressure regulation valve 1212a may be actuated to fluidically connect the vacuum source to the first gas/liquid separation module 111a. Negative pressure from the vacuum source may permeate through the membrane 350 of the first gas/liquid separation module 111a and may provide a suction force in the first flow path 1226a. The suction force in the first flow path 1226a may draw the sample 1252a into the first flow path 1226a and into the retentate portion of the first gas/liquid separation module 111a.

When the retentate portion of the first gas/liquid separation module 111a is at least partially filled, the multi-port selection valve 106 may be actuated to fluidically connect the secondary flow path 1228 to the intermediate flow path 1227a. The flow path valve 1208a may be actuated to open the intermediate flow path 1227a. The peristaltic pump 1302 may be operated to draw the sample 1352a into the intermediate flow path 1227a. The peristaltic pump 1302 may pull the sample 1352a downstream from the multi-port selection valve 106 and at least partially into the secondary flow path 1228.

In some embodiments, the process may include a sampling step, wherein the flow path valve 1208a is closed and the multi-port selection valve 106 is actuated to fluidically connect the secondary flow path 1228 to an air port or cleaning agent port. The peristaltic pump 1302 may pull the air or cleaning agent (and any of the sample 1352a disposed within the secondary flow path 1228) through the peristaltic pump 1302 and the downstream flow path 1350 to a waste receptacle. The amount of sample 1352a can now be accurately controlled, as the entire intermediate flow path 1227a may be filled with the sample 1352a and none of the secondary flow path 1228 is filled with the sample 1352a. Any pumping of the intermediate flow path 1227a will immediately fill the secondary flow path 1228, and thus, the amount of sample 1352a drawn into the secondary flow path 1228 can be accurately measured/controlled via the peristaltic pump 1302. For instance, FIG. 13B illustrates the aseptic sampling system 1300 in a prepared state.

The multi-port selection valve 106 may actuate to fluidically connect the intermediate flow path 1227a to the secondary flow path 1228 and the flow path valve 1208a may be opened. The peristaltic pump 1302 may pull the sample 1352a into the secondary flow path 1228, through the peristaltic pump 1302, and through the downstream flow path 1350 to a downstream analyzer. After a controlled amount of the sample 1352a is pulled through the multi-port selection valve 106, the flow path valve 1208a may be closed, and the multi-port selection valve 106 may be actuated to fluidically connect to an air port. The peristaltic pump 1302 may pull the sample 1352a remaining in the secondary flow path 1228 to the downstream analyzer.

In some embodiments, the process may include a clearing step. The pressure regulation valve 1212 may be actuated to fluidically connect the air source to the respective gas/liquid separation module 111a, 111b. The air source may urge the sample 1352a located within the first flow path 1226a back toward the reservoir 130a. The flow path valve 1208a may actuate to open the intermediate flow path 1227a and the multi-port selection valve 106 may actuate to fluidically connect the intermediate flow path 1227a to the secondary flow path 1228. The remaining sample 1352a may be drawn from the intermediate flow path 1227a to the secondary flow path 1228 and out of the downstream flow path 1350 via the peristaltic pump 1302.

In some embodiments, the above process may be repeated for the second bioreactor (i.e., reservoir 130b connected to the first flow path 1226b and the intermediate flow path 1227b). In some embodiments, the second bioreactor may be primed while the above process is occurring. For example, FIG. 13C illustrates the aseptic sampling system 1300 with the second bioreactor primed for aseptic sampling of the sample 1352b.

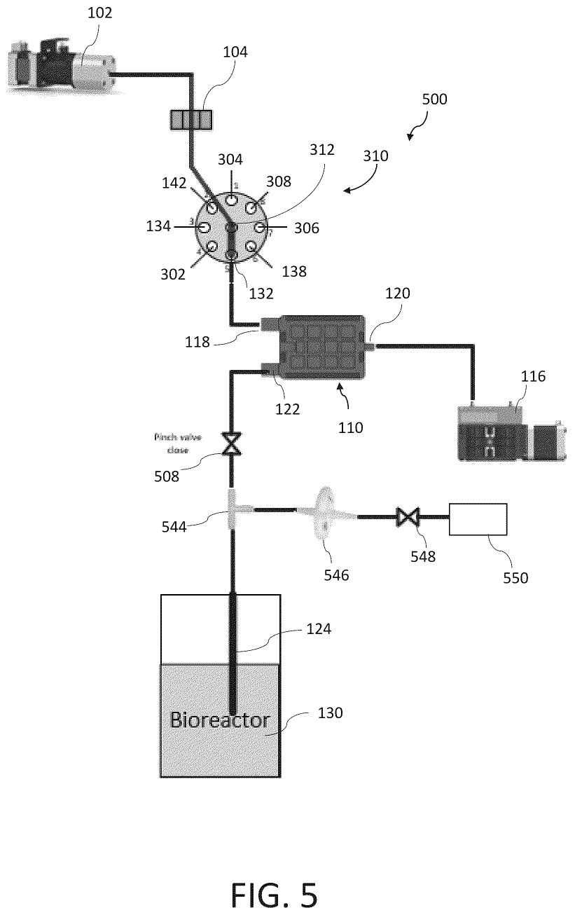

FIG. 5 illustrates a diagrammatic view of an exemplary aseptic sampling system 500 including gas/liquid separator outside of the aseptic zone. The advantage of this embodiment is that the gas/liquid separator 110 does not need to be sterilized via an autoclave process. The aseptic sampling system 500 may include a tee 544 configured to fluidically connect a pressurized gas source 550 to the flow path. The pressurized gas source 550 may be selectively connected and disconnected to the flow path via a valve 548, and optionally through a filter 546 to sterilize the pressurized gas supplied to the flow path. A pressure regulation system may operate the pressurized gas source 550 to push fluid material from the tee 544 back toward and preferably into the reservoir 130. In the embodiment shown in FIG. 5, a vacuum source 116 may be fluidically connected to gas/liquid separator 110, and operated to draw sample from the reservoir 130 through the flow path to the separation chamber with a negative pressure applied to gas/liquid separator 110 as described in other embodiments hereof. The negative pressure may be effective to degas (i.e., separate gas from liquid) the sample through a semi-permeable membrane 350 in gas/liquid separator 110. The system 500 in FIG. 5 may be operated to push the sample back into the reservoir 130 via the pressurized gas source 550 and tee 544, rather than relying solely upon sufficient positive pressure at a permeate side of the membrane 350 to push the sample along the flow path.

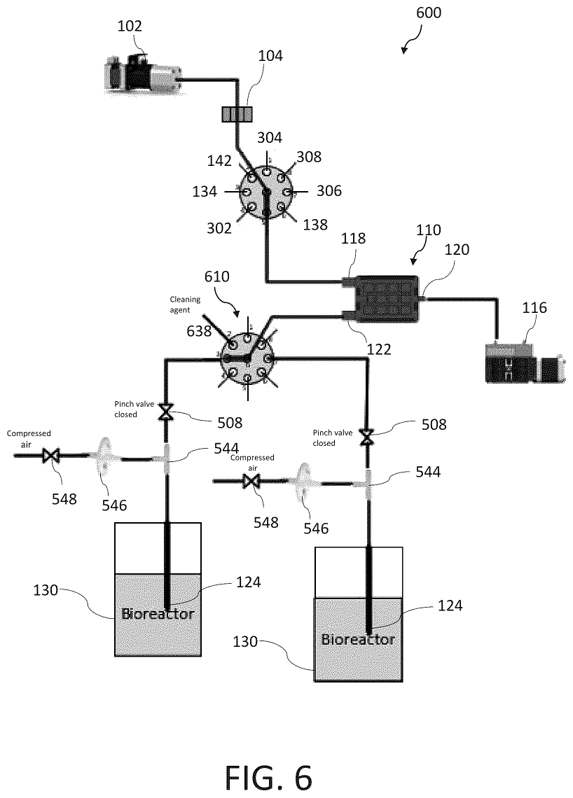

FIG. 6 illustrates a diagrammatic view of an exemplary aseptic sampling system 600 including multiple reservoirs on a single gas/liquid separator. The advantage of this embodiment is that the gas/liquid separator 110 does not need to be sterilized via an autoclave process and the system is configured to draw samples from multiple different reservoirs/bioreactors. In some embodiments, two or more reservoirs may be fluidically connected to a multi-port selection valve 610. The multi-port selection valve 610 may include a cleaning agent port 638 configured to introduce a cleaning agent into the flow path between the multi-port selection valve 610 and the gas/liquid separator 110. The gas/liquid separator 110 may be configured to pull the cleaning agent through the flow path via the vacuum source 116.

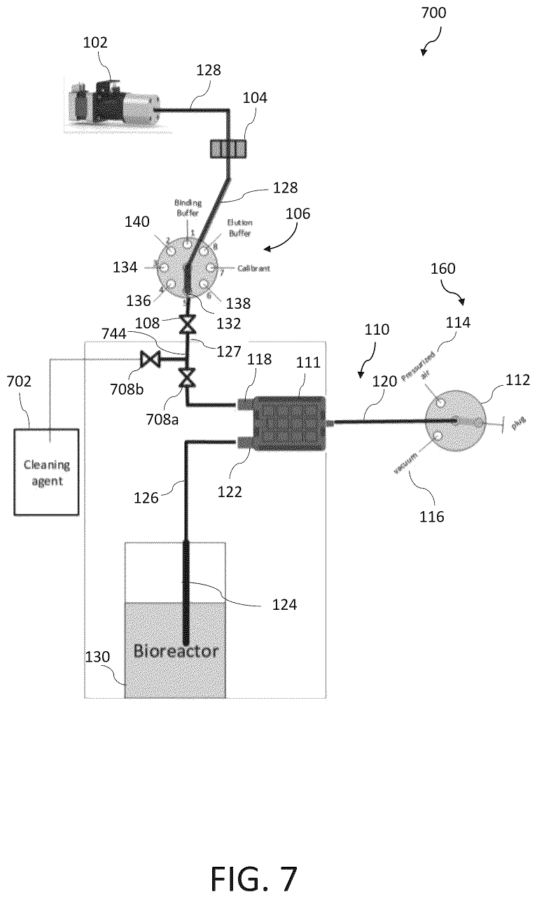

FIG. 7 illustrates a diagrammatic view of an exemplary aseptic sampling system 700 including a cleaning agent reservoir 702 configured to sterilize a portion of the intermediate flow path 127. In some embodiments, the pump 102 may pull the cleaning agent from the cleaning agent reservoir through the intermediate flow path 127. The cleaning agent may be pulled into the flow path between the valve 708b and the multi-port selection valve 106 to sanitize (i.e., sterilize or aseptically clean) that portion of the flow path. Valve 708a is closed to prevent the cleaning agent from flowing into the gas/liquid separator 110. The pump 102 may aspirate the cleaning agent from the intermediate flow path 127 through the multi-port selection valve 106 to the second flow path 128. Valve 708a may be open during the aspiration of the pump 102. In some cases, cleaning agent may remain between the valve 708a and tee 744. The pressurized gas source 114 of the gas/liquid separator may be used to push the remaining cleaning agent from the tee 744 to the cleaning agent reservoir 702.

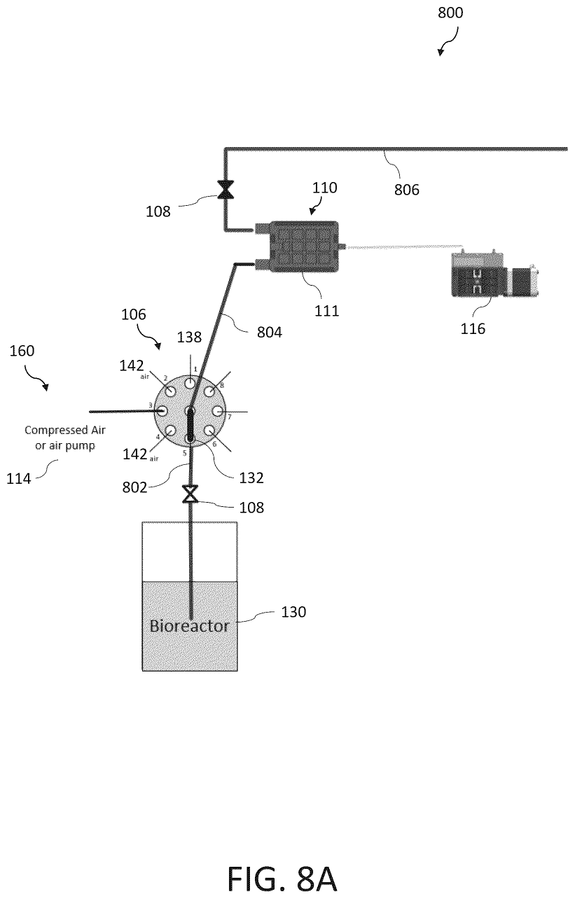

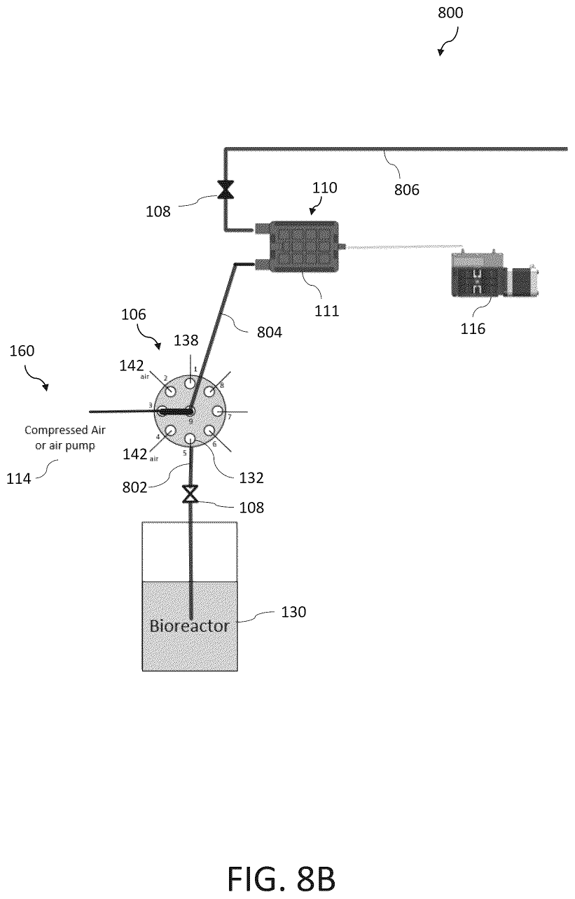

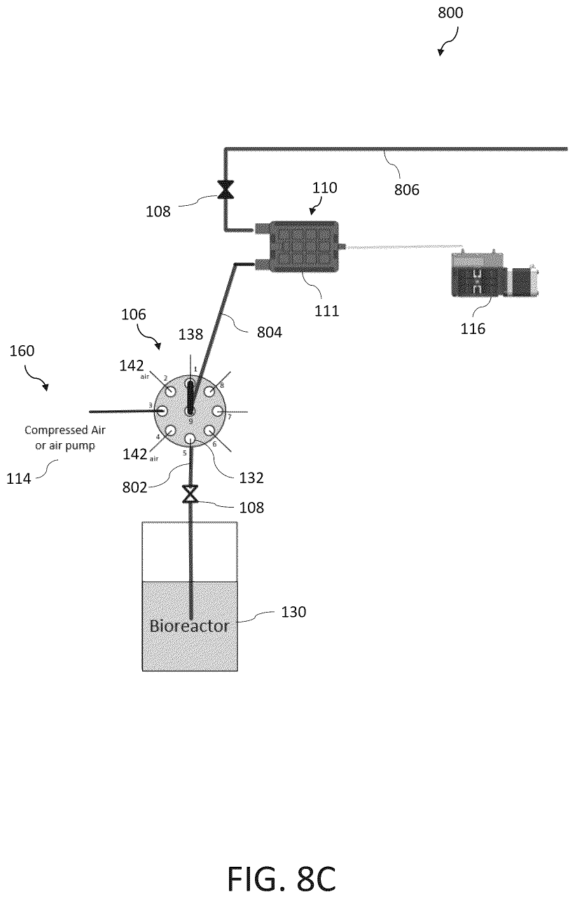

FIGS. 8A-8C illustrate an exemplary aseptic sampling system 800 including a multi-port selection valve 106 located between the reservoir 130 and the gas/liquid separator 110, according to some embodiments. The pressurized gas source 114 may fluidically connect to the multi-port selection valve 106 located between the reservoir 130 and the gas/liquid separator 110. A first flow path 820 may fluidically connect the reservoir 130 to the multi-port selection valve 106 and an intermediate flow path 804 may fluidically connect the multi-port selection valve 106 to the gas/liquid separator 110. The vacuum source 116 may draw a sample from the reservoir 130 to the intermediate flow path 804 when the sample port 132 is fluidically connected to the vacuum source 116 via the multi-port selection valve 106. In some embodiments, the multi-port selection valve 106 may include a plurality of sample ports 132 each fluidically connectable to a reservoir. The multi-port selection valve 106 may selectively connect the pressurized gas source 114 to the intermediate flow path 804 to push any sample located within the intermediate flow path 804 to a second flow path 806 located between the gas/liquid separator 110 and a downstream analyzer (not shown). Thus, the aseptic sampling system 800 may operate as a push-and-pull system, with the vacuum source 116 pulling sample into the intermediate flow path 804 with negative pressure, and the pressurized air source pushing the sample toward a downstream analyzer with a positive pressure. The aseptic sampling system 800 may switch between pulling the sample (with the vacuum source 116) and pushing the sample (with the pressurized gas source 114) via actuation of the multi-port selection valve 106.

In some embodiments, before a sample is drawn from the reservoir 130, the multi-port selection valve 106 may connect to the pressurized gas source 114 (see e.g., FIG. 8B). The pressurized gas source 114 may push gas, such as air, through the intermediate flow path 804, through the gas/liquid separator 110, and through the second flow path 806 to clear the flow path of any residual air, cleaning agent, sample, or other material located within the flow path. The multi-port selection valve 106 may acuate to fluidically connect the intermediate flow path 804 to the air port 142 to release pressure within the flow path. If the multi-port selection valve 106 transitioned directly from the pressurized gas source 114 to the reservoir 130, the positive pressure in the flow path may push the sample into the reservoir 130.

In some embodiments, to draw a sample from the reservoir 130, the multi-port selection valve 106 may connect the reservoir 130 to the intermediate flow path 804 (see e.g., FIG. 8A). The vacuum source 116 may generate a low pressure condition within a permeate portion of the gas/liquid separator 110. The low pressure condition in the permeate portion may permeate through the membrane 350 and create a low pressure condition in the retentate portion of the gas/liquid separation module 111. The low pressure condition permeating through the membrane 350 may provide a suction force to the intermediate flow path 804 and the first flow path 802. Thus, a sample may be pulled from the reservoir 130 to the first flow path 802, through the multi-port selection valve 106, to the intermediate flow path 804. In some embodiments, the tubing between the multi-port selection valve 106 and the gas/liquid separator 110 may be selected so that the internal volume of the intermediate flow path 804, plus the internal volume of the retentate portion of the gas/liquid separator 110, is the maximum sample volume that can be delivered to the downstream analyzer per cycle.

In some embodiments, to push the drawn sample from the intermediate flow path 804 and the retentate portion of the gas/liquid separator 110, the multi-port selection valve 106 actuates to fluidically connect to the pressurized gas source 114 (see e.g., FIG. 8B). The pressurized gas source 114 may push the sample located within the intermediate flow path 804 and the gas/liquid separator 110 to the downstream analyzer (via the second flow path 806). In some embodiments, the pressurized gas source 114 may provide a regulated gas pressure to control a sample delivery speed. For instance, dependent upon viscosity and/or sample volume, a higher or lower pressure may be required to deliver the sample to the downstream analyzer. In some cases, if gas pressure is too high, the sample segment may be disturbed by the pressure, i.e., broken up, overly-agitated, aerated, or damaged. Thus, in some embodiments, the pressurized gas source 114 may include a pump configured to control the flow rate of the sample. In some embodiments, the flow rate may be controlled to allow the vacuum source 116 to degas the sample as it passes through the separation chamber. For instance, if only a portion of the drawn sample can fit within the separation chamber, the gas/liquid separator may continue to degas the sample as the pressurized gas source 114 pushes the sample through.

In some embodiments, the intermediate flow path 804 may include a gas/liquid separator (not shown) integrated into the flow path. The integrated gas/liquid separator may generate a negative pressure condition in the intermediate flow path 804 and pull dissolved gasses through a gas-permeable, liquid impermeable membrane.

In some embodiments, to clean the flow path, the multi-port selection valve 106 may acuate to fluidically connect the cleaning agent port 138 to a cleaning agent reservoir (see e.g., FIG. 8C). The vacuum source 116 may generate the low pressure condition to draw the cleaning agent into the intermediate flow path 804 and/or the gas/liquid separator 110. The multi-port selection valve 106 may then actuate to fluidically connect the pressurized gas source 114 to the intermediate flow path 804 and push the cleaning agent through the gas/liquid separator 110 and the second flow path 806. The pressurized gas source 114 may push out substantially all of the cleaning agent from the flow path, and thus, the aseptic sampling system 800 may be sterilized and ready for the next sample withdrawal cycle.

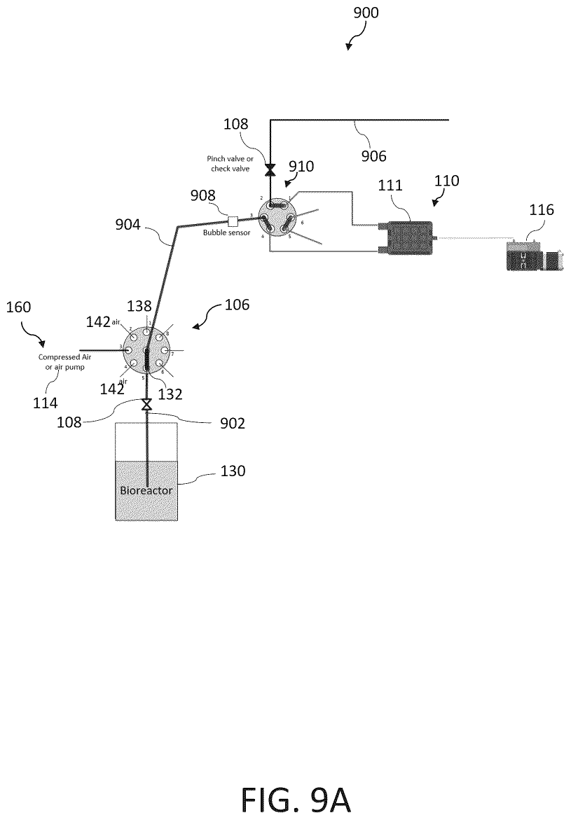

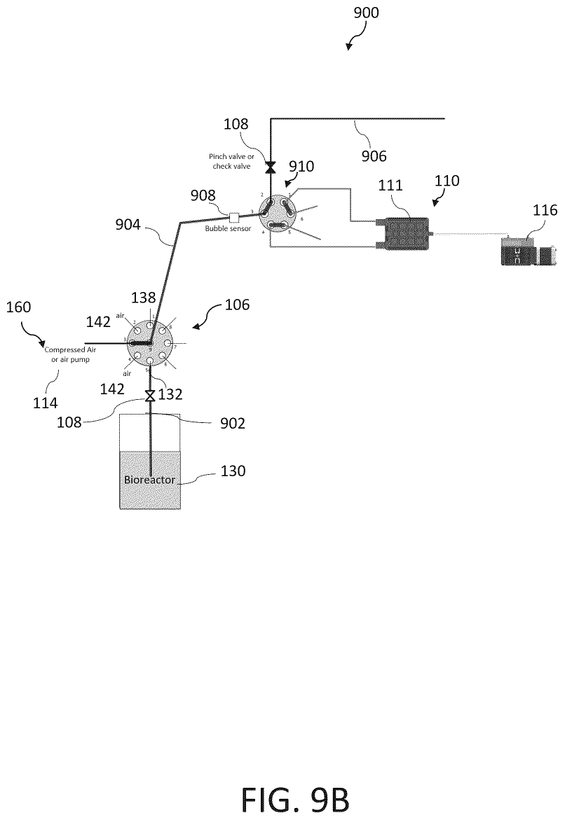

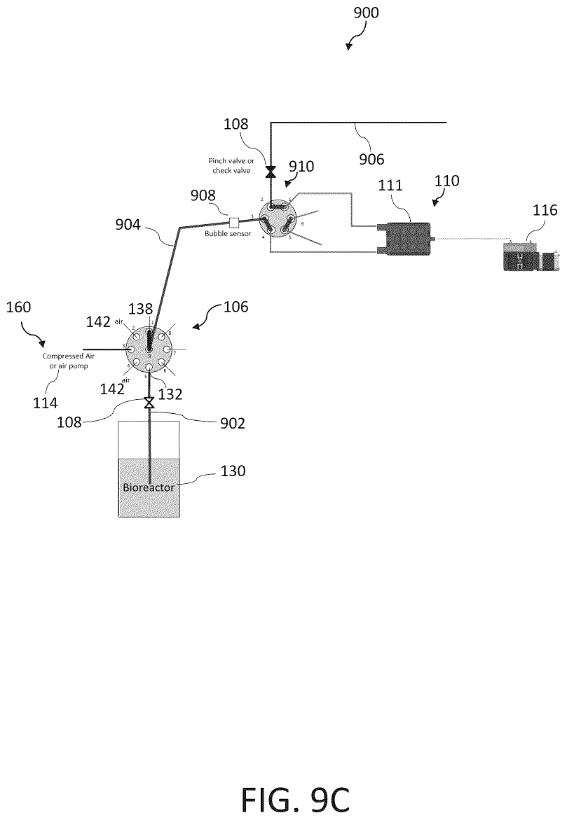

FIGS. 9A-C illustrate an exemplary aseptic sampling system 900 including a multi-port selection valve 106 located between the reservoir 130 and the gas/liquid separator 110, and an injection valve 910, according to some embodiments. The aseptic sampling system 900 may selectively connect and disconnect the gas/liquid separator 110 from the multi-port selection valve 106 via actuation of the injection valve 910. Such configuration may be beneficial, as the gas/liquid separator 110 may contribute to sample carryover between sampling cycles and may be difficult to sterilize with the cleaning agent. The aseptic sampling system 900 allows a selective bypass of the gas/liquid separator 110 when drawing a sample from the reservoir 130 to a downstream analyzer. The aseptic sampling system 900 may include a first flow path 902 fluidically connecting the reservoir 130 to the multi-port selection valve 106. The aseptic sampling system may include an intermediate flow path 904 fluidically connecting the multi-port selection valve 106 to the injection valve 910. In some embodiments, a fluid sensor 908 may be located on the intermediate flow path 904. The fluid sensor 908 may sense when a sample has reached the fluid sensor 908 on the intermediate flow path 904 and generate a signal to control the injection valve 910 accordingly. For example, when a sample has reached the fluid sensor 908, the fluid sensor 908 may signal the injection valve 910 to actuate to disconnect from the gas/liquid separator 110, thus, preventing the sample from entering the gas/liquid separator 110. The aseptic sampling system 900 may include a second flow path 906 fluidically connecting the injection valve 910 to a downstream analyzer.

In some embodiments, to draw a sample from the first flow path 902 to the intermediate flow path 904, the injection valve 910 fluidically connects the gas/liquid separator 110 to the multi-port selection valve 106 (see e.g., FIG. 9A). The gas/liquid separator 110 may generate a suction force to pull sample from the reservoir 130 to the intermediate flow path 904. The sample may fill an internal volume of the intermediate flow path 904 until it reaches the fluid sensor 908.

In some embodiments, the fluid sensor 908 may generate a signal in response to fluid (e.g., sample) passing by the fluid sensor 908. The signal may be communicated to a processor, CPU, or user interface (not shown), and control actuation of the multi-port selection valve 106 and/or the injection valve 910. For instance, in some cases, if a sample is detected at the fluid sensor 908, the signal may cause the injection valve 910 to fluidically disconnect from the gas/liquid separator 110 and cause the multi-port selection valve 106 to fluidically connect to the pressurized gas source 114 (see e.g., FIG. 9B). Thus, the sample may bypass the gas/liquid separator 110 by traveling directly from the intermediate flow path 904 to the second flow path 906. In some embodiments, the fluid sensor 908 may measure one or more characteristics of the sample, including but not limited to: entrained air percent by volume, viscosity, transparency, acoustic impedance of ultrasound, and/or other gas-in-fluid detection characteristics known in the art. In some embodiments, if the one or more characteristics is indicative of gas-in-fluid above a threshold amount, the fluid sensor 908 (or CPU) may selectively route the sample to the gas/liquid separator 110 to degas the sample. If the one or more characteristics is indicative of gas-in-fluid below a threshold amount, the aseptic sampling system 900 may bypass the gas/liquid separator 110 via actuating the multi-port selection valve 106 and/or injection valve 910 as shown in FIG. 9B. In some embodiments, an additional cleaning agent can be introduced to the gas/liquid separator 110 through one or more ports on the injection valve 910.

In some embodiments, the aseptic sampling system 900 may be sterilized by a cleaning agent introduced through the cleaning agent port 138 (see e.g., FIG. 9C). The injection valve may fluidically connect the gas/liquid separator 110 to the multi-port selection valve 106 and a negative pressure generated by the vacuum source 116 may generate suction to draw the cleaning agent into the intermediate flow path 904. In some embodiments, the cleaning agent may selectively bypass the gas/liquid separator 110. Bypassing the gas/liquid separator 110 may depend on whether the sample bypassed the gas/liquid separator 110, with the cleaning agent following the same flow path as the sample.

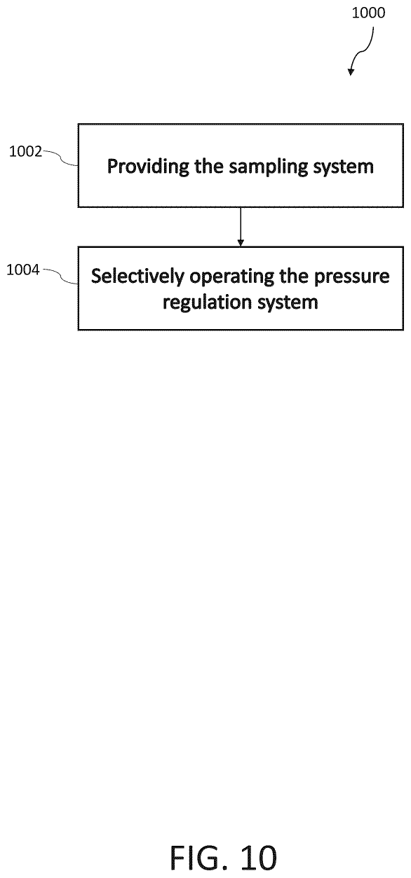

FIG. 10 illustrates an exemplary flow chart of a method 1000 of automated aseptic sampling. The method 1000 includes providing the sampling system 1002. The sampling system may include any of the embodiments described and/or illustrated above. The sampling system may include a gas/liquid separator having a separation chamber. The gas/liquid separator includes a membrane separating the separation chamber into a retentate portion and a permeate portion. The gas/liquid separator includes a pressure regulation system fluidically connected to the permeate portion of the separation chamber.

The method 1000 includes selectively operating the pressure regulation system 1004. The pressure regulation system may be operated in a first mode to establish a first gas pressure condition in the permeate portion of the separation chamber to draw a sample of the liquidous material from a reservoir through a first flow path to the retentate portion of the separation chamber. The pressure regulation system may be operated in a second mode to establish a second condition in the permeate portion of the separation chamber to push the sample from the permeate portion of the separation chamber, and, in some embodiments, through the first flow path toward the reservoir.

In some embodiments, the membrane is gas-permeable and liquid impermeable. The first mode may be effective to remove a gas from the sample through the membrane. The method 1000 may further include pumping the sample through the first flow path with a displacement pump in fluidic connection to the first flow path. The displacement pump may be fluidically connected to the first flow path through a multiple-port selection valve that is selectively adjustable among a plurality of positions. A first position fluidically connects the first flow path to a second flow path, and a second position fluidically disconnects the first flow path from the second flow path. The second position fluidically connects the second flow path to a third flow path. The method may further include pumping the sample through the third flow path when the selection valve is in the second position. The third flow path may be fluidically connected to an analysis system.

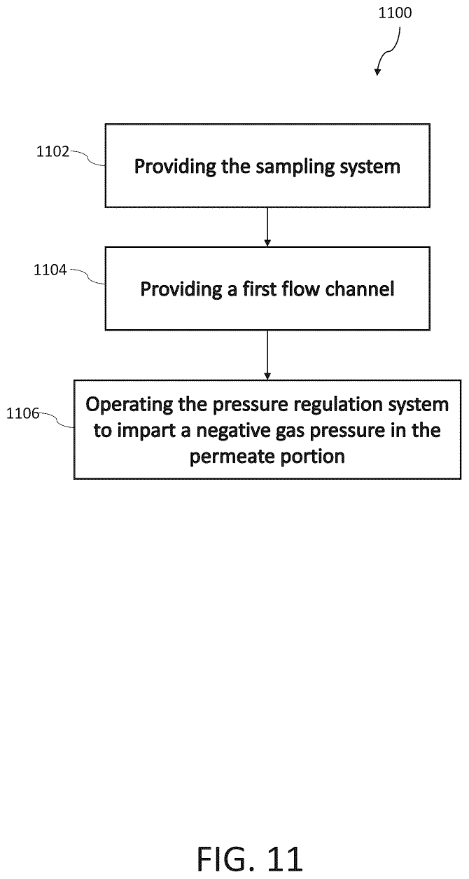

FIG. 11 illustrates an exemplary flow chart of a method of automated aseptic sampling. The method 1100 includes providing a sampling system 1102. The sampling system includes a gas/liquid separator having a separation chamber. The separation chamber is separated into a retentate portion and a permeate portion by a membrane. The gas/liquid separator includes a pressure regulation system configured to control a pressure in the permeate portion. The method 1100 includes providing a first flow path 1104. The first flow path connects the separation chamber to a reservoir containing the liquidous material. The method 1100 includes operating the pressure regulation system to impart a negative gas pressure in the permeate portion 1106. The negative pressure in the permeate portion is effective to pull a sample of the liquidous material from the reservoir to the retentate portion of the separation chamber. The negative pressure in the permeate portion is effective to remove a gas from the sample through the membrane.

In some embodiments, the method 1100 further includes operating the pressure regulation system to impart a positive pressure in the permeate portion. The positive pressure in the permeate portion is effective to push the sample from the retentate portion of the gas/liquid separation chamber toward the reservoir. The pressure regulation system may include a vacuum source fluidically connected to the permeate portion, a pressurized air source fluidically connected to the permeate portion, and a control valve. The control valve may selectively connect and/or selectively disconnect the pressurized air source and the vacuum source to the permeate portion of the separation chamber. The method 1100 may further include providing a second flow path fluidically connecting the retentate portion of the separation chamber to a displacement pump and pumping the sample through the second flow path from the retentate portion of the separation chamber toward the displacement pump. The first flow path may be fluidically connected to the second flow path through a multiple-port selection valve. Each of the plurality of ports may be selectively connectable to the displacement pump.

While the disclosure has been described with reference to an exemplary embodiment(s), it will be understood by those skilled in the art that various changes may be made, and equivalents may be substituted for elements thereof without departing from the scope of the embodiment(s). In addition, many modifications may be made to adapt a particular situation or material to the teachings of the embodiment(s) without departing from the essential scope thereof. Therefore, it is intended that the disclosure is not limited to the disclosed embodiment(s), but that the disclosure will include all embodiments falling within the scope of the appended claims. Various examples have been described. These and other examples are within the scope of the following claims.

What is claimed is:

1. A method of sampling a liquidous material with a sampling system, the method comprising:
providing the sampling system having a gas/liquid separator, the gas/liquid separator including:
a separation chamber,
a membrane separating the separation chamber into a retentate portion and a permeate portion, wherein the membrane is gas-permeable and liquid-impermeable, and
a pressure regulation system fluidically connected to the permeate portion of the separation chamber, wherein the pressure regulation system is adapted to control a gas pressure in the permeate portion; and
selectively operating the pressure regulation system in:
a first mode to establish a first gas pressure condition in the permeate portion of the separation chamber effective to draw a sample of the liquidous material from a reservoir through a first flow channel to the retentate portion of the separation chamber, and
a second mode to establish a second gas pressure condition in the permeate portion of the separation chamber effective to push the sample from the retentate portion of the separation chamber through the first flow channel.

2. The method of claim 1, wherein the first gas pressure condition is effective to remove a gas from the sample through the membrane.

3. The method of claim 1 further comprising:
pumping the sample through the first flow channel with a displacement pump in fluidic connection to the first flow channel.

4. The method of claim 3, wherein the displacement pump is fluidically connected to the first flow channel through a multiple-port selection valve that is selectively adjustable among a plurality of positions, wherein a first position fluidically connects the first flow channel to a second flow channel, and wherein a second position fluidically disconnects the first flow channel from the second flow channel, the second position fluidically connecting the second flow channel to a third flow channel.

5. The method of claim 4, further comprising:
pumping the sample through the third flow channel when the selection valve is in the second position.

6. The method of claim 5, wherein the third flow channel is fluidically connected to an analysis system.

7. The method of claim 1, including operating the pressure regulation system in a third mode to establish a third pressure condition in the permeate portion of the degassing chamber effective to cause substantially no fluid flow in the first fluid channel.

8. A method of sampling a liquidous material, the method comprising:
providing a sampling system having a gas/liquid separator, the gas/liquid separator including:
a separation chamber having a retentate portion, a permeate portion, and
a membrane separating the retentate portion and the permeate portion, wherein the membrane is gas-permeable and impermeable to the liquidous material, and
a pressure regulation system configured to control a pressure in the permeate portion;
providing a first flow channel fluidically connecting the separation chamber to a reservoir containing the liquidous material; and
operating the pressure regulation system to impart a negative gas pressure in the permeate portion, wherein the negative gas pressure in the permeate portion is effective to pull a sample of the liquidous material from the reservoir to the retentate portion of the separation chamber; and wherein the negative gas pressure in the permeate portion is effective to remove a gas from the sample through the membrane.

9. The method of claim 8, further comprising:
operating the pressure regulation system to impart a positive gas pressure in the permeate portion, wherein the positive gas pressure in the permeate portion is effective to push the sample from the retentate portion of the separation chamber.

10. The method of claim 9, wherein the pressure regulation system includes a vacuum source fluidically connected to the permeate portion, a pressurized air source fluidically connected to the permeate portion, and a control valve.

11. The method of claim 10, including operating the control valve to selectively connect or disconnect at least one of the pressurized air source and the vacuum source to the permeate portion of the separation chamber.

12. The method of claim 8, further comprising:
providing a second flow channel fluidically connecting the retentate portion of the separation chamber to a displacement pump; and
pumping the sample through the second flow channel from the retentate portion of the separation chamber toward the displacement pump.

13. The method of claim 12, wherein the retentate portion of the separation chamber is fluidically connected to the displacement pump along the second flow channel through a selection valve, the selection valve including a plurality of ports, wherein each of the plurality of ports is selectively fluidically connectable to the displacement pump.

14. The method of claim 13, wherein the plurality of ports includes an analyzer port fluidically connected to an analyzer flow channel.

15. The method of claim 13, further comprising:
pumping the sample through the analyzer flow channel to an analysis system.

16. The method of claim 9, further comprising:
operating the pressure regulation system to impart a neutral gas pressure in the permeate portion, wherein the neutral gas pressure in the permeate portion is effective to cause substantially no fluid flow in the first flow channel.

17. A sampling system, comprising:
a reservoir containing a liquidous material;
a gas/liquid separator including:
a separation chamber having a retentate portion, a permeate portion, and a membrane separating the retentate portion and the permeate portion, wherein the membrane is gas-permeable and impermeable to the liquidous material, and
a pressure regulation system configured to control a gas pressure in the permeate portion;
a first flow channel fluidically connecting the reservoir to the retentate portion of the gas/liquid separator;
a displacement pump;
a multiple port selection valve; and
a second flow channel fluidically connecting the displacement pump to the selection valve, wherein the selection valve is selectively adjustable among a plurality of positions, with a first position fluidically connecting the first flow channel to the second flow channel,
wherein the pressure regulation system is configured to selectively effect a negative gas pressure in the permeate portion, wherein the negative gas pressure in the permeate portion is effective to pull a sample of the liquidous material from the reservoir to the retentate portion of the separation chamber, and wherein the pressure regulation system is configured to selectively effect a positive gas pressure in the permeate portion, wherein the positive gas pressure in the permeate portion is effective to push the sample of the liquidous material from the retentate portion of the gas/liquid separation chamber to the reservoir.

18. The sampling system of claim 17, wherein the negative pressure in the permeate portion is effective to remove a gas from the sample through the membrane.

19. The sampling system of claim 17, further comprising:
a probe for obtaining the sample from the reservoir; and
a second valve for selectively closing and opening the first flow channel between the selection valve and the probe.

20. The sampling system of claim 17, wherein a second position of the selection valve fluidically disconnects the first flow channel from the second flow channel, and fluidically connects the second flow channel to a third flow channel.

21. The sampling system of claim 17, wherein the third flow channel fluidically connects the selection valve to one or more of an analysis system, a fluid source, and a waste receptacle.

22. The sampling system of claim 17, further comprising an intermediate flow path fluidically connectable to the retentate portion and the multiple port selection valve, wherein the intermediate flow path includes a flow path valve to selectively open and close the intermediate flow path.
